(19) United States
(12) Patent Application Publication
(10) Pub. No.: US 2018/0098933 A1
(43) Pub. Date: Apr. 12, 2018

(54) COMPOSITION FOR TREATMENT OF HAIR LOSS AND REGROWTH STIMULATION

(71) Applicant: Nina Elya Ltd., Ashkelon (IL)

(72) Inventor: Richard Rani WEISSBORT, Ashkelon (IL)

(73) Assignee: Nina Elya Ltd., Ashkelon (IL)

(21) Appl. No.: 15/836,964

(22) Filed: Dec. 11, 2017

Related U.S. Application Data
(63) Continuation-in-part of application No. PCT/IL2016/050607, filed on Jun. 9, 2016.
(60) Provisional application No. 62/173,986, filed on Jun. 11, 2015.

(51) Int. Cl. A61K 8/9794; A61K 8/9789; A61K 8/63; A61K 8/49; A61K 8/46; A61K 8/58; A61K 8/67; A61K 8/19; A61K 8/23; A61K 8/60; A61Q 7/00

(52) U.S. Cl. CPC A61K 8/9794; A61K 2800/5922; A61K 8/63; A61K 8/498; A61K 8/466; A61K 8/58; A61K 8/678; A61K 8/673; A61K 8/46; A61K 8/19; A61K 8/67; A61K 8/23; A61K 8/602; A61Q 7/00; A61K 8/9789

(57) ABSTRACT

This invention is directed to a composition for treating and preventing hair loss in male and female, for regrowth stimulation of hair in male and female, for replenishing the previously colored hair in male and female, and for stimulation of healthy and strong finger nail growth, wherein the composition is a nutrition supplement.

### CROSS REFERENCE TO RELATED APPLICATIONS

This Application is a Continuation-In-Part of International Application Number PCT/IL2016/050607, filed 9 Jun. 2016, which claims priority of U.S. Provisional Ser. No. 62/173,986 filed 11 Jun. 2015; which are incorporated in their entirety herein by reference.

### FIELD OF THE INVENTION

This invention is directed to a composition for treating and preventing hair loss for male and female, for regrowth stimulation of hair, for replenishing the previously colored hair in male and female, and for the stimulation of healthy and strong finger nail growth, wherein the composition is a nutrition supplement.

### BACKGROUND OF THE INVENTION

Hairs are elongated filamentous structures projecting from the hair follicles that have an almost unique ability to regenerate. Hairs vary in size and color from coarse, highly-pigmented terminal hair that is produced by large-diameter long follicles, to tiny almost invisible vellus hair produced by miniaturized follicles.

The growth of hair is a cyclical process made up of successive phases. These phases include growth phase (anagen) during which the hair is produced, a short regressive phase (catagen), and a resting phase (telogen) during which a new hair is formed and the original hair is shed. The length of time that the follicle is in each phase and the size of the follicle is regulated by androgens.

In non-balding scalp, more than 90% of scalp hair is in anagen. However, in men with Adrogenic alopecia, shortening of the anagen phase lead the hair follicles to undergo progressive miniaturization, shrinking from terminal to vellus-like hair which contributes to a decrease of visible hair over affected areas of the scalp.

Hair loss, also known as alopecia, affects both males and females, and may be caused by genetic factors, auto-immune disorders, chemotherapy, aging, local or systemic disease, etc. Men susceptible to male pattern baldness usually, experience the onset sometime in their 20's and it becomes more common as they age.

Alopecia may occur in various parts of the human body, such as scalp, face, limbs, and trunk, and may be generally grouped in the following categories: male or female pattern baldness (e.g., androgenic alopecia), toxic alopecia (e.g. hair loss due to chemotherapy), alopecia aerata (i.e., hair loss occurs in demarcated areas), and scarring alopecia (e.g., hair loss due to inflammation and tissue destruction).

Androgenetic alopecia (AGA) is the most common type of alopecia. Androgenetic alopecia is a patterned, progressive loss of an excessive amount of hair from the scalp. Significant Androgenetic alopecia occurs in 50% of men by the age of fifty and 50% of women by the age of sixty. The key factor in the pathogenesis of AGA is dihydrotestosterone (DHT), a metabolite of testosterone converted by the enzyme 5α-reductase. DHT was shown to shorten the anagen phase of the hair cycle and mediate miniaturization of the follicles that produce progressively finer hair.

Alopecia areata is a nonscarring form of hair loss which occurs in humans and other species and is thought to be due to an inflammatory reaction caused by autoimmune response directed against the anagen stage hair follicle structure, other factors may influence hair loss, many of which are temporary include stress of an illness or major surgery, medicines, such as those used in chemotherapy, blood thinners, antidepressants, excessive amounts of vitamin A and certain disease states like diabetes.

A variety of treatments that inhibit the activity of 5α-reductase were shown to promote hair regrowth in clinical studies.

These include the orally delivered FDA approved prescription drug Propecia (finasteride 1 mg) as well as several botaniceuticals. Unfortunately, sexual dysfunction side effects were found in post-marketing studies of Propecia. Other FDA approved treatments for AGA that do not specifically target the DHT mechanism include the over-the-counter topically administered drug Rogaine (minoxidil 5%) and the LaserComb phototherapy device. Hair transplant surgery currently done by follicular unit graft transplantation is an expensive option for hair restoration. In this technique grafts containing hair follicles that are genetically resistant to balding are transplanted to the bald scalp

There exists a need for (i) hair loss treatment in male and female; (ii) for hair regrowth stimulation compositions in male and female; (iii) replenishing the previously colored hair in male and female and (iv) for promoting healthy and strong finger nail growth using nutrition supplements with minimal side effects and/or health risks.

### SUMMARY OF THE INVENTION

In one embodiment, this invention provides a nutrition supplement composition comprising serenoa repens, stephania tetrandra, at least one amino acid, at least one vitamin and at least one minerals. In another embodiment, the composition further comprises phytosterol. In another embodiment, the composition further comprises isoflavonoid. In another embodiment, the composition further comprises taurine and bambusa.

In one embodiment, this invention provides a method of stimulating healthy and strong finger nail growth in male and female comprising administering the composition of this invention.

In one embodiment, this invention provides a method of treating, preventing, inhibiting or any combination thereof of hair loss; hair growth stimulation; replenishing the previously colored hair; stimulation of healthy and strong finger nail growth; or any combination thereof in a subject, comprising administering the composition of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a vertax photograph of a subject after using 6 month of Formulation B.

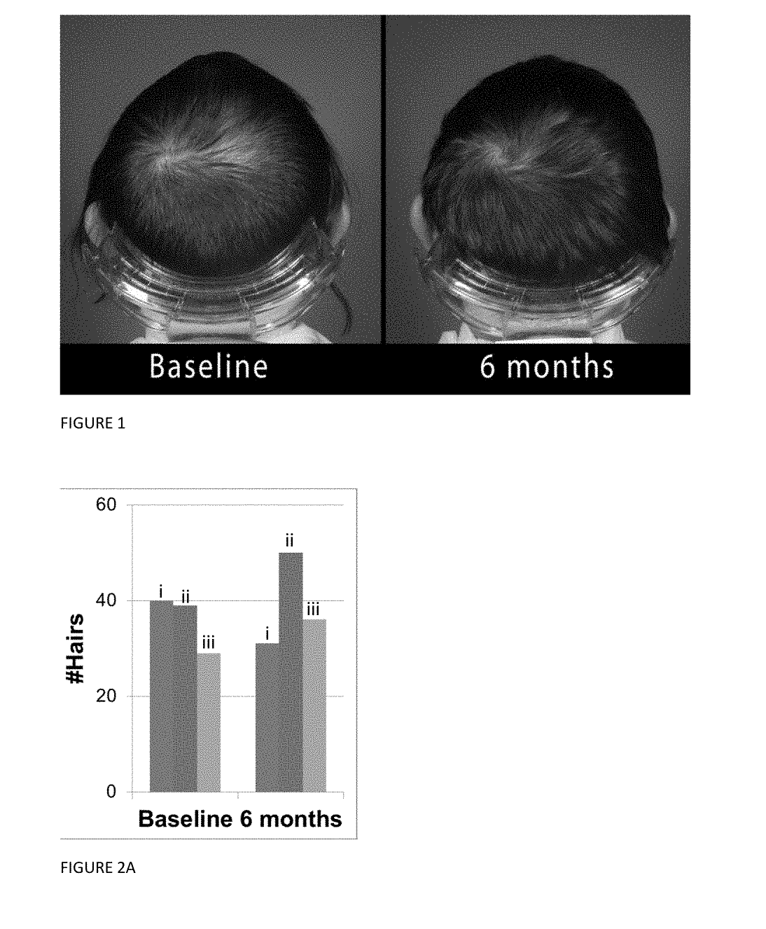

FIGS. 2A and 2B presents results after six month of treatment using Formulation A of the terminal hair versus the vellus hair FIG. 2A presents a graph with hair count results (i) is Vellus hair counts (ii) is light terminal hair count and (iii) is dark terminal count. It is observed that the vellus hair counts (i) are decreased, while terminal hairs counts (ii) and (iii) are increased. FIG. 2B is a macro photograph for hair-count.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, the composition of this invention includes plant extracts, dried herbs, amino acid, vitamins and minerals which are safe with no side effects. The composition of this invention is safe with no side effects.

In one embodiment, this invention is directed to a nutrition supplement composition comprising serenoa repens, cepharanthine, amino acid, vitamins and minerals.

In one embodiment, this invention is directed to a nutrition supplement composition comprising serenoa repens, stephania extract, amino acid, vitamins and minerals.

In one embodiment, this invention is directed to a nutrition supplement composition comprising serenoa repens, amino acid, vitamins and minerals.

In one embodiment, this invention provides a nutrition supplement composition comprising serenoa repens, stephania tetrandra, at least one amino acid, at least one vitamin and at least one minerals.

In another embodiment, amino acid is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. Each represents a separate embodiment of this invention. In another embodiment, the composition of this invention comprises between 1 to 3 different amino acids. In another embodiment, the composition comprises 1, 2, 3, 4 different amino acids. Each represents a different embodiment of this invention.

In another embodiment, vitamins are selected from vitamin A, vitamin B₁, vitamin B₂, vitamin B₃, vitamin B₅, vitamin B₆, vitamin B₇, vitamin B₉, vitamin B₁₂, vitamin C, vitamin D, vitamin E and vitamin K. Each represents a separate embodiment of this invention. In another embodiment, the composition of this invention comprises between 1 to 10 different vitamins In another embodiment, the composition comprises 1, 2, 3, 4, 5, 6, 7 different vitamins Each represents a different embodiment of this invention.

In one embodiment, this invention is directed to a nutrition supplement composition comprising serenoa repens, cepharanthine, taurine, bambusa, amino acid, vitamins and minerals.

In one embodiment, this invention is directed to a nutrition supplement composition comprising serenoa repens, cepharanthine, taurine, bambusa, cysteine, vitamins and minerals.

In one embodiment, this invention is directed to a nutrition supplement composition comprising serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium.

In one embodiment, this invention is directed to a nutrition supplement composition comprising serenoa repens, stephania, amino acid, vitamins, minerals and phytosterol.

In one embodiment, this invention provides a nutrition supplement composition comprising serenoa repens, stephania tetrandra, at least one amino acid, at least one vitamin and at least one minerals and phytosterol.

In another embodiment, phytosterols are selected from □-sitosterole, campesterol, cholesterol, stigmasterol and stigmastanol.

In one embodiment, this invention is directed to a nutrition supplement composition comprising serenoa repens, stephania, amino acid, vitamins, minerals, phytosterol and isoflavonoid.

In one embodiment, this invention provides a nutrition supplement composition comprising serenoa repens, stephania tetrandra, at least one amino acid, at least one vitamin and at least one minerals, phytosterol and isoflavonoid.

In another embodiment, non limiting examples of used isoflavonoids include: genistin, genisteine, (S)-equol, daidzein, Liquiritigenin, glycinol and isoflavane.

In one embodiment, this invention is directed to a nutrition supplement composition comprising serenoa repens, stephania, amino acid, vitamins, minerals phytosterol, taurine and bambusa.

In one embodiment, this invention is directed to a nutrition supplement composition comprising serenoa repens, stephania tetrandra, amino acid, vitamins, minerals phytosterol, taurine and bambusa.

In one embodiment, this invention is directed to a nutrition supplement composition comprising serenoa repens, stephania, amino acid, vitamins, minerals, phytosterol, isoflavonoid, taurine and bambusa.

In one embodiment, this invention is directed to a nutrition supplement composition comprising serenoa repens, stephania tetrandra, at least one amino acid, at least one vitamin, at least one mineral, phytosterol, isoflavonoid, taurine and bambusa.

In one embodiment, this invention is directed to a nutrition supplement composition comprising serenoa repens, stephania, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium.

In one embodiment, this invention is directed to a nutrition supplement composition comprising serenoa repens, stephania tetranda, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium.

In one embodiment, this invention is directed to a nutrition supplement composition comprising serenoa repens, stephania, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium, β-sitosterole and iron fumarate.

In one embodiment, this invention is directed to a nutrition supplement composition comprising serenoa repens, stephania tetranda, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium, β-sitosterole and iron fumarate

In one embodiment, this invention is directed to a nutrition supplement composition comprising serenoa repens, taurine, bambusa, amion acid, vitamins and minerals.

In one embodiment, this invention is directed to a nutrition supplement composition comprising serenoa repens, taurine, bambusa, cysteine, vitamins and minerals.

In one embodiment, this invention is directed to a nutrition supplement composition comprising serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium.

In another embodiment, the composition of this invention comprises stephania, wherein the stephania is selected from Stephania Tetrandra, Stephania Cepharantha Hayata, Stephania erecta Craib, Stephania rotunda Lour, Stephania rotunda Lour, Stephania sinica Diels and Stephania suberosa L. L. Forman.

In one embodiment, this invention is directed to a nutrition supplement composition comprising serenoa repens, stephania, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium, β-sitosterole and Genistin.

In another embodiment, stephania is selected from Stephania Tetrandra, Stephania Cepharantha Hayata, Stephania erecta Craib, Stephania rotunda Lour, Stephania rotunda Lour, Stephania sinica Diels and Stephania suberosa L. L. Forman.

In one embodiment, this invention is directed to a nutrition supplement composition comprising serenoa repens, Stephania Tetranda, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium, β-sitosterole and Genistin.

In one embodiment, the composition of this invention comprises serenoa repens (extract and/or dry herb). In another embodiment, the serenoa repens is a dry herb. In another embodiment, the serenoa repens is an extract of serenoa repens containing 85-90% fatty acids and sterols, with an abundance of carotenoids, lipases, tannin, sugars and fatty acids like caprylic acid, palmitic acid, oleic acid and beta sitosterol.

In one embodiment, the composition of this invention comprises cepharanthine. In another embodiment, the cepharanthine is pure. In another embodiment, the cepharanthine is distilled or isolated from stephania cepharantha hayata. In another embodiment, the composition of this invention does not include cepharanthine.

In one embodiment, this invention is directed to a nutrition supplement composition comprising serenoa repens, stephania extract containing or not containing cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium.

A genus stephania belongs to family menispermaceae, a large family of about 65 genera and 350 species [Journal of Ethnopharmacology, 2010, 132, 369-383]. Non limiting examples of stephania extract which includes cepharanthine include: Stephania Cepharantha Hayata, Stephania erecta Craib, Stephania rotunda Lour, Stephania rotunda Lour, Stephania sinica Diels and Stephania suberosa L. L. Forman. In another embodiment, stephania tetrandra or extract thereof does not include cepharanthine.

In another embodiment, Stephania Cepharantha Hayata extract contains inter alia stephaoxocanine, stephaoxocanidine, romorphinane, cepharanthine, cepharanoline, steponine, isotetrandrine, berbamine, stecepharine, dehydroreticuline, magnoflorine, menisperine, oblongine, cyclanoline, cis-N-methylcapaurine, sinomenine, cephamerphinanine, D-glucopyranoside, 2′-N-methylisotetrandrine, N-methyl stesakine chloride, stesakine 9-O-beta-D-glucoside, N-methylasimilobine-2-O-beta-D-glucopyranoside, cephamonine, aromoline, zippelianine, cepharamine, aknadinine, aknadicine, cephatonine, (−)-cycleanine, obamegine, berbamine, isocorydine, anolobine, cotypalline, stepharine, (+)-reticuline, obaberine, homoaromoline, fangchinoline, tetrandrine and cephamuline. In another embodiment, the Stephania Cepharantha Hayata extract is a root extract.

In another embodiment, Stephania Erecta Craib extract contains inter alia cepharanthine, (+)-2-N-methyltelobine, (+)-1,2 dehydrotelobine, (+)-2-nor-isotetrandrine, (+)-isotetrandrine, (+)-2-nor-thalrugosine, (+)-thalrugosine, (+)-homoaromoline, (+)-stephibaberine, (+)-dephnandrine, (+)-2-nor-cepharanthine, (+)-nor-obaberine and (+)-obaberine.

In another embodiment, Stephania Rotunda Lour extract contains inter alia cepharamine, cycleanine, (+)-stepharine, (−)-tetrahydropalmatine, coclaurine, dehassiline, stepholidine, corynoxidine, stepharotudine, cepharanthine, fangchinoline, 5-hydroxy-6,7-dimethoxy-3,4-dihydroisoquinolin-1(2H)-one, thaicanine 4-O-beta-D-glucoside and (−)-thaicanine N-oxide (4-hydroxycorynoxidine)

In another embodiment, Stephania sinica Diels extract contains inter alia cepharanthine, runanine and beta-sitosterol.

In another embodiment, Stephania suberosa L. L. Forman extract contains inter alia (+)-cepharanthine, (+)-2-norcepharanthine, (+)-cepharanthine 2 alpha-beta -N-oxide, (+)-stephasubine, (+)-norstephasubine, stephasubimine, (−)-tetrahydrostephabine, (−)-kikemanine, (−)-stephabinamine, tephabine, stephnubine, (−)-discretine, 8-oxypseudopalmatine, (−)-tetrahydropalmatrubine, (−)-stepholidine, (−)-capaurimine, pseudopalmatine, (−)-coreximine, stephaphylline, (−)-ctytenchine, (−)-xylopinine and nordelavaine.

A bambusa is a large genus of clumping bamboos, containing about 147 species, including but not limited to the following species: bamboosa affinis, bamboosa alcooa, bambusa chungii, bamboosa jacobsii, bamboosa multiplex, bamboosa spinose, bamboosa tulda and bamboosa ventricosa. In another embodiment, bamboosa vulgaris contains inter alia cellulose, pentosans, lignin, ash and silica.

In another embodiment, bamboosa arundinacea contains inter alia Oxalic acid, reducing sugar, resins, waxes, HCN, benzoic acid, diferuloyl arabinoxylanhexasaccharide, diferuloyl oligosaccharide, (5,5′-di-(diferul-9,9′-dioyl)-[α-Larabinofuranosyl-(1→3)-O-β-D-xylopyranosyl-9 (1→4)-D-xylopyranose] (taxiphyllin), arginine, cysteine, histidine, isoleucine, leucine, lysine, methionine, phenylamine, threonine, valine, tyrosine, niacin, riboflavin, thiamine, Protein, gluteline, lysine, methionine, betain, cholin, proteolytic enzyme, nuclease and urease (Chatterjee and Pakrashi, 2001).

In one embodiment, the nutrition supplement composition for male and female comprises:
between 20-40 mg serenoa repens, in another embodiment, about 25 mg serenoa repens, in another embodiment, about 30 mg serenoa repens;
between 2-10 mg cepharanthine, in another embodiment, about 2 mg cepharanthine, in another embodiment, about 5 mg cepharanthine;
between 20-40 mg tocopherol (vitamin E), in another embodiment, about 25 mg tocopherol (vitamin E), in another embodiment, about 30 mg tocopherol (vitamin E);
between 20-40 mg taurine, in another embodiment, about 25 mg taurine, in another embodiment, about 30 mg taurine;
between 20-40 mg pyridoxine, in another embodiment, about 25 mg pyridoxine, in another embodiment, about 30 mg pyridoxine;
between 15-35 mg bambusa, in another embodiment, about 30 mg bambusa; in another embodiment, about 25 mg bambusa;
between 15-35 mg zinc picolinate, in another embodiment, about 30 mg zinc picolinate, in another embodiment, about 25 mg zinc picolinate, in another embodiment, about 22 mg zinc picolinate;
between 10-30 mg cysteine, in another embodiment, about 20 mg cysteine, in another embodiment, about 25 mg cysteine;
between 10-30 mg thiamine (vitamin B1), in another embodiment, about 20 mg thiamine (vitamin B1), in another embodiment, about 25 mg thiamine (vitamin B1);
between 10-30 mg pantothenic acid (vitamin B5), in another embodiment, about 20 mg pantothenic acid (vitamin B5), in another embodiment, about 25 mg pantothenic acid (vitamin B5);
between 10-30 mg riboflavin (vitamin B2), in another embodiment, about 20 mg riboflavin (vitamin B2), in another embodiment, about 15 mg riboflavin (vitamin B2);
between 1-5 mg copper, in another embodiment, about 1 mg copper, in another embodiment, about 1.5 mg copper, in another embodiment, about 1 mg copper, in another embodiment, about 2 mg copper;
between 0.1-1 mg cobalamin (vitamin B12), in another embodiment, about 0.1 mg cobalamin (vitamin B12), in another embodiment, about 0.2 mg cobalamin (vitamin B12), in another embodiment, about 0.3 mg cobalamin (vitamin B12);
between 0.1-1 mg biotine (vitamin H), in another embodiment, about 0.1 mg biotine (vitamin H), in another embodiment, about 0.2 mg biotine (vitamin H), in another embodiment, about 0.3 mg biotine (vitamin H); and
between 0.1-1 mg selenium, in another embodiment, about 0.1 mg selenium, in another embodiment, about 0.2 mg selenium, in another embodiment, about 0.3 mg selenium.

In another embodiment, the nutrition supplement composition for male and female comprises:
30 mg serenoa repens;
2 mg cepharanthine;
30 mg tocopherol (vitamin E);
30 mg taurine;
30 mg pyridoxine;
25 mg bambusa;
22 mg zinc picolinate;
20 mg cysteine;
20 mg thiamine (vitamin B1)
20 mg pantothenic acid (vitamin B5);
15 mg riboflavin (vitamin B2);
1.5 mg copper;
0.3 mg cobalamin (vitamin B12);
0.3 mg biotine (vitamin H); and
0.1 mg selenium.

In one embodiment, the nutrition supplement composition for male and female comprises:
between 200 to 400 mg serenoa repens, in another embodiment, about 200 mg serenoa repens, in another embodiment, about 300 mg serenoa repens;
between 50 to 250 mg stephania extract, in another embodiment, about 100 mg stephania extract in another embodiment, about 200 mg stephania extract.
between 20-40 mg tocopherol (vitamin E), in another embodiment, about 25 mg tocopherol (vitamin E), in another embodiment, about 30 mg tocopherol (vitamin E);
between 20-40 mg taurine, in another embodiment, about 25 mg taurine, in another embodiment, about 30 mg taurine;
between 20-40 mg pyridoxine, in another embodiment, about 25 mg pyridoxine, in another embodiment, about 30 mg pyridoxine;
between 15-35 mg bambusa, in another embodiment, about 30 mg bambusa; in another embodiment, about 25 mg bambusa;
between 15-35 mg zinc picolinate, in another embodiment, about 30 mg zinc picolinate, in another embodiment, about 25 mg zinc picolinate, in another embodiment, about 22 mg zinc picolinate;
between 10-30 mg cysteine, in another embodiment, about 20 mg cysteine, in another embodiment, about 25 mg cysteine;
between 10-30 mg thiamine (vitamin B1), in another embodiment, about 20 mg thiamine (vitamin B1), in another embodiment, about 25 mg thiamine (vitamin B1);
between 10-30 mg pantothenic acid (vitamin B5), in another embodiment, about 20 mg pantothenic acid (vitamin B5), in another embodiment, about 25 mg pantothenic acid (vitamin B5);
between 10-30 mg riboflavin (vitamin B2), in another embodiment, about 20 mg riboflavin (vitamin B2), in another embodiment, about 15 mg riboflavin (vitamin B2);
between 1-5 mg copper, in another embodiment, about 1 mg copper, in another embodiment, about 1.5 mg copper, in another embodiment, about 1 mg copper, in another embodiment, about 2 mg copper;
between 0.1-1 mg cobalamin (vitamin B12), in another embodiment, about 0.1 mg cobalamin (vitamin B12), in another embodiment, about 0.2 mg cobalamin (vitamin B12), in another embodiment, about 0.3 mg cobalamin (vitamin B12);
between 0.1-1 mg biotine (vitamin H), in another embodiment, about 0.1 mg biotine (vitamin H), in another embodiment, about 0.2 mg biotine (vitamin H), in another embodiment, about 0.3 mg biotine (vitamin H); and
between 0.1-1 mg selenium, in another embodiment, about 0.1 mg selenium, in another embodiment, about 0.2 mg selenium, in another embodiment, about 0.3 mg selenium.

In another embodiment, the nutrition supplement composition for male and female comprises:
300 mg serenoa repens;
100 mg stephania;
30 mg tocopherol (vitamin E);
30 mg taurine;
30 mg pyridoxine;
25 mg bambusa;
22 mg zinc picolinate;
20 mg cysteine;
20 mg thiamine (vitamin B1)
20 mg pantothenic acid (vitamin B5);
15 mg riboflavin (vitamin B2);
1.5 mg copper;
0.3 mg cobalamin (vitamin B12);
0.3 mg biotine (vitamin H); and
0.1 mg selenium.

In one embodiment, the nutrition supplement composition for male comprises:
between 20 to 40 mg serenoa repens, in another embodiment, about 20 mg serenoa repens, in another embodiment, about 30 mg serenoa repens;
between 5 to 15 mg stephania extract, in another embodiment, about 5 mg stephania extract, in another embodiment, about 10 mg stephania extract, in another embodiment, the stephania extract is selected from Stephania Tatrandra, Stephania Cepharantha Hayata, Stephania erecta Craib, Stephania rotunda Lour, Stephania rotunda Lour, Stephania sinica Diels and Stephania suberosa L. L. Forman. Each represents a separate embodiment.
between 40-60 mg of β-sitosterole, in another embodiment, about 45 mg β-sitosterole, in another embodiment about 50 mg β-sitosterole.
between 20-40 mg tocopherol (vitamin E), in another embodiment, about 25 mg tocopherol (vitamin E), in another embodiment, about 30 mg tocopherol (vitamin E);
between 200-400 mg taurine, in another embodiment, about 250 mg taurine, in another embodiment, about 300 mg taurine;
between 20-40 mg pyridoxine, in another embodiment, about 25 mg pyridoxine, in another embodiment, about 30 mg pyridoxine;
between 15-35 mg bambusa, in another embodiment, about 30 mg bambusa; in another embodiment, about 25 mg bambusa;
between 15-35 mg zinc picolinate, in another embodiment, about 15 mg zinc picolinate, in another embodiment, about 25 mg zinc picolinate, in another embodiment, about 22 mg zinc picolinate;
between 10-30 mg cysteine, in another embodiment, about 20 mg cysteine, in another embodiment, about 25 mg cysteine;
between 10-30 mg thiamine (vitamin B1), in another embodiment, about 20 mg thiamine (vitamin B1), in another embodiment, about 25 mg thiamine (vitamin B1);
between 10-30 mg pantothenic acid (vitamin B5), in another embodiment, about 20 mg pantothenic acid (vitamin B5), in another embodiment, about 25 mg pantothenic acid (vitamin B5);
between 10-30 mg riboflavin (vitamin B2), in another embodiment, about 20 mg riboflavin (vitamin B2), in another embodiment, about 15 mg riboflavin (vitamin B2);
between 1-5 mg copper, in another embodiment, about 1 mg copper, in another embodiment, about 1.5 mg copper, in another embodiment, about 1 mg copper, in another embodiment, about 2 mg copper;
between 0.1-1 mg cobalamin (vitamin B12), in another embodiment, about 0.1 mg cobalamin (vitamin B12), in another embodiment, about 0.2 mg cobalamin (vitamin B12), in another embodiment, about 0.3 mg cobalamin (vitamin B12);
between 0.1-1 mg biotine (vitamin H), in another embodiment, about 0.1 mg biotine (vitamin H), in another embodiment, about 0.2 mg biotine (vitamin H), in another embodiment, about 0.3 mg biotine (vitamin H);
between 0.1-1 mg selenium, in another embodiment, about 0.1 mg selenium, in another embodiment, about 0.2 mg selenium, in another embodiment, about 0.3 mg selenium; and
between 10-25 mg iron fumarate, in another embodiment about 10 mg iron fumarate; in another embodiment about 15 mg iron fumarate.

In another embodiment, the nutrition supplement composition for male comprises:
10 mg stephania, wherein said stephania is selected from Stephania Tetrandra, Stephania Cepharantha Hayata, Stephania erecta Craib, Stephania rotunda Lour, Stephania rotunda Lour, Stephania sinica Diels or Stephania suberosa L. L. Forman. Each represents a separate embodiment;
30 mg serenoa repens;
50 mg β-sitosterole
30 mg tocopherol (vitamin E);
300 mg taurine;
30 mg pyridoxine;
20 mg bambusa;
22 mg zinc picolinate;
20 mg cysteine;
20 mg thiamine (vitamin B1)
20 mg pantothenic acid (vitamin B5);
15 mg riboflavin (vitamin B2);
1.5 mg copper;
0.3 mg cobalamin (vitamin B12);
0.3 mg biotine (vitamin H);
0.1 mg selenium; and
15 mg iron fumarate.

In one embodiment, the nutrition supplement composition for male comprises:
between 20 to 40 mg serenoa repens, in another embodiment, about 20 mg serenoa repens, in another embodiment, about 25 mg serenoa repens, in another embodiment, about 30 mg serenoa repens, in another embodiment, about 35 mg serenoa repens, in another embodiment, about 40 mg serenoa repens;
between 5 to 15 mg stephania extract, in another embodiment, about 5 mg stephania extract, in another embodiment, about 10 mg stephania extract, in another embodiment, about 15 mg stephania extract, in another embodiment, the stephania extract is selected from Stephania Tatrandra, Stephania Cepharantha Hayata, Stephania erecta Craib, Stephania rotunda Lour, Stephania rotunda Lour, Stephania sinica Diels and Stephania suberosa L. L. Forman Each represents a separate embodiment;
between 40-60 mg of β-sitosterole, in another embodiment, about 40 mg β-sitosterole, in another embodiment, about 45 mg β-sitosterole, in another embodiment about 50 mg β-sitosterole, in another embodiment, about 55 mg β-sitosterole, in another embodiment, about 60 mg β-sitosterole;
between 20-40 mg tocopherol (vitamin E), in another embodiment, about 20 mg tocopherol (vitamin E), in another embodiment, about 25 mg tocopherol (vitamin E); in another embodiment, about 30 mg tocopherol (vitamin E), in another embodiment, about 35 mg tocopherol (vitamin E), in another embodiment, about 4 mg tocopherol (vitamin E);
between 200-400 mg taurine, in another embodiment, about 200 mg taurine, in another embodiment, about 250 mg taurine, in another embodiment, about 300 mg taurine, in another embodiment, about 290 mg taurine, in another embodiment, about 350 mg taurine, in another embodiment, about 400 mg taurine;
between 20-40 mg pyridoxine, in another embodiment, about 20 mg pyridoxine, in another embodiment, about 25 mg pyridoxine, in another embodiment, about 30 mg pyridoxine, in another embodiment, about 35 mg pyridoxine, in another embodiment, about 40 mg pyridoxine;
between 15-35 mg bambusa, in another embodiment, about 15 mg bambusa, in another embodiment, about 20 mg bambusa, in another embodiment, about 25 mg bambusa, in another embodiment, about 30 mg bambusa, in another embodiment, about 35 mg bambusa;
between 15-35 mg zinc picolinate, in another embodiment, about 15 mg zinc picolinate, in another embodiment, about 20 mg zinc picolinate, in another embodiment, about 25 mg zinc picolinate, in another embodiment, about 22 mg zinc picolinate, in another embodiment, about 30 mg zinc picolinate, in another embodiment, about 35 mg zinc picolinate;
between 10-30 mg cysteine, in another embodiment, about 10 mg cysteine, in another embodiment, about 15 mg cysteine,in another embodiment, about 20 mg cysteine, in another embodiment, about 25 mg cysteine, in another embodiment, about 30 mg cysteine;
between 10-30 mg thiamine chloride (vitamin B1), in another embodiment, about 10 mg thiamine chloride (vitamin B1), in another embodiment, about 15 mg thiamine chloride (vitamin B1), in another embodiment, about 20 mg thiamine chloride (vitamin B1), in another embodiment, about 25 mg thiamine chloride (vitamin B1); in another embodiment, about 30 mg thiamine chloride (vitamin B1);
between 10-30 mg pantothenic acid (vitamin B5), in another embodiment, about 10 mg pantothenic acid (vitamin B5), in another embodiment, about 15 mg pantothenic acid (vitamin B5),in another embodiment, about 20 mg pantothenic acid (vitamin B5), in another embodiment, about 25 mg pantothenic acid (vitamin B5), in another embodiment, about 30 mg pantothenic acid (vitamin B5),
between 10-30 mg riboflavin (vitamin B2), in another embodiment, about 10 mg riboflavin (vitamin B2), in another embodiment, about 15 mg riboflavin (vitamin B2), in another embodiment, about 20 mg riboflavin (vitamin B2), in another embodiment, about 25 mg riboflavin (vitamin B2), in another embodiment, about 30 mg riboflavin (vitamin B2);
between 1-5 mg copper, in another embodiment, about 1 mg copper, in another embodiment, about 1.5 mg copper, in another embodiment, about 2 mg copper, in another embodiment, about 2.5 mg copper, in another embodiment, about 3 mg copper, in another embodiment, about 3.5 mg copper, in another embodiment, about 4 mg copper, in another embodiment, about 4.5 mg copper, in another embodiment, about 5 mg copper;
between 0.1-1 mg cobalamin (vitamin B12), in another embodiment, about 0.1 mg cobalamin (vitamin B12), in another embodiment, about 0.2 mg cobalamin (vitamin B12), in another embodiment, about 0.3 mg cobalamin (vitamin B12), in another embodiment, about 0.4 mg cobalamin (vitamin B12), in another embodiment, about 0.5 mg cobalamin (vitamin B12), in another embodiment, about 0.6 mg cobalamin (vitamin B12), in another embodiment, about 0.7 mg cobalamin (vitamin B12), in another embodiment, about 0.8 mg cobalamin (vitamin B12), in another embodiment, about 1 mg cobalamin (vitamin B12);
between 0.1-1 mg biotine (vitamin H), in another embodiment, about 0.1 mg biotine (vitamin H), in another embodiment, about 0.2 mg biotine (vitamin H), in another embodiment, about 0.3 mg biotine (vitamin H), in another embodiment, about 0.4 mg biotine (vitamin H), in another embodiment, about 0.5 mg biotine (vitamin H), in another embodiment, about 0.6 mg biotine (vitamin H), in another embodiment, about 0.7 mg biotine (vitamin H), in another embodiment, about 0.8 mg biotine (vitamin H), in another embodiment, about 0.9 mg biotine (vitamin H), in another embodiment, about 1 mg biotine (vitamin H);
between 0.1-1 mg selenium, in another embodiment, about 0.1 mg selenium, in another embodiment, about 0.2 mg selenium, in another embodiment, about 0.3 mg selenium, in another embodiment, about 0.4 mg selenium, in another embodiment, about 0.5 mg selenium, in another embodiment, about 0.6 mg selenium, in another embodiment, about 0.7 mg selenium, in another embodiment, about 0.8 mg selenium, in another embodiment, about 0.9 mg selenium, in another embodiment, about 1 mg selenium;
between 5-25 mg ferrous bisglycinate, in another embodiment 5 mg ferrous bisglycinate, in another embodiment 10 mg ferrous bisglycinate, in another embodiment 15 mg ferrous bisglycinate, in another embodiment 20 mg ferrous bisglycinate, in another embodiment 25 mg ferrous bisglycinate.

In another embodiment, the nutrition supplement composition for male comprises:
10 mg stephania, wherein said stephania is selected from Stephania Tetrandra, Stephania Cepharantha Hayata, Stephania erecta Craib, Stephania rotunda Lour, Stephania rotunda Lour, Stephania sinica Diels or Stephania suberosa L. L. Forman Each represents a separate embodiment;
30 mg serenoa repens;
50 mg β-sitosterole
30 mg tocopherol (vitamin E);
290 mg taurine;
30 mg pyridoxine;
35 mg bambusa;
22 mg zinc picolinate;
20 mg cysteine;
20 mg thiamine (vitamin B1)
20 mg pantothenic acid (vitamin B5);
15 mg riboflavin (vitamin B2);
1.5 mg copper;
0.3 mg cobalamin (vitamin B12);
0.3 mg biotine (vitamin H);
0.1 mg selenium; and
15 mg ferrous bisglycinate.

In one embodiment, the nutrition supplement composition for female comprises:
between 20 to 40 mg serenoa repens, in another embodiment, about 20 mg serenoa repens, in another embodiment, about 30 mg serenoa repens;
between 5 to 15 mg stephania extract, in another embodiment, about 5 mg stephania extract, in another embodiment, about 10 mg stephania extract, in another embodiment, the stephania extract is selected from Stephania Tatrandra, Stephania Cepharantha Hayata, Stephania erecta Craib, Stephania rotunda Lour, Stephania rotunda Lour, Stephania sinica Diels and Stephania suberosa L. L. Forman; Each represents a separate embodiment;
between 20-30 mg of β-sitosterole, in another embodiment, about 20 mg β-sitosterole, in another embodiment about 25 mg β-sitosterole.
between 20-40 mg tocopherol (vitamin E), in another embodiment, about 25 mg tocopherol (vitamin E), in another embodiment, about 30 mg tocopherol (vitamin E);
between 200-400 mg taurine, in another embodiment, about 250 mg taurine, in another embodiment, about 300 mg taurine;
between 20-40 mg pyridoxine, in another embodiment, about 25 mg pyridoxine, in another embodiment, about 30 mg pyridoxine;
between 15-35 mg bambusa, in another embodiment, about 20 mg bambusa; in another embodiment, about 25 mg bambusa;
between 15-35 mg zinc picolinate, in another embodiment, about 15 mg zinc picolinate, in another embodiment, about 22 mg zinc picolinate, in another embodiment, about 22 mg zinc picolinate;
between 10-30 mg cysteine, in another embodiment, about 20 mg cysteine, in another embodiment, about 25 mg cysteine;
between 10-30 mg thiamine (vitamin B1), in another embodiment, about 20 mg thiamine (vitamin B1), in another embodiment, about 25 mg thiamine (vitamin B1);
between 10-30 mg pantothenic acid (vitamin B5), in another embodiment, about 20 mg pantothenic acid (vitamin B5), in another embodiment, about 25 mg pantothenic acid (vitamin B5);
between 10-30 mg riboflavin (vitamin B2), in another embodiment, about 20 mg riboflavin (vitamin B2), in another embodiment, about 15 mg riboflavin (vitamin B2);
between 1-5 mg copper, in another embodiment, about 1 mg copper, in another embodiment, about 1.5 mg copper, in another embodiment, about 1 mg copper, in another embodiment, about 2 mg copper;
between 0.1-1 mg cobalamin (vitamin B12), in another embodiment, about 0.1 mg cobalamin (vitamin B12), in another embodiment, about 0.2 mg cobalamin (vitamin B12), in another embodiment, about 0.3 mg cobalamin (vitamin B12);
between 0.1-1 mg biotine (vitamin H), in another embodiment, about 0.1 mg biotine (vitamin H), in another embodiment, about 0.2 mg biotine (vitamin H), in another embodiment, about 0.3 mg biotine (vitamin H);
between 0.1-1 mg selenium, in another embodiment, about 0.1 mg selenium, in another embodiment, about 0.2 mg selenium, in another embodiment, about 0.3 mg selenium; and
between 20-40 mg genisteine, in another embodiment about 30 mg genisteine; in another embodiment about 35 mg genisteine.

In another embodiment, the nutrition supplement composition for female comprises:
10 mg stephania, wherein said stephania is selected from Stephania Tetrandra, Stephania Cepharantha Hayata, Stephania erecta Craib, Stephania rotunda Lour, Stephania rotunda Lour, Stephania sinica Diels or Stephania suberosa L. L. Forman; Each represents a separate embodiment;
30 mg serenoa repens;
25 mg β-sitosterole
30 mg tocopherol (vitamin E);
300 mg taurine;
30 mg pyridoxine;
20 mg bambusa;
22 mg zinc picolinate;
20 mg cysteine;
20 mg thiamine (vitamin B1)
20 mg pantothenic acid (vitamin B5);
15 mg riboflavin (vitamin B2);
1.5 mg copper;
0.3 mg cobalamin (vitamin B12);
0.3 mg biotine (vitamin H);
0.1 mg selenium; and
30 mg genisteine.

In one embodiment, the nutrition supplement composition for female comprises:
between 20 to 40 mg serenoa repens, in another embodiment, about 20 mg serenoa repens, about 25 mg serenoa repens, in another embodiment, about 30 mg serenoa repens, about 35 mg serenoa repens, about 40 mg serenoa repens;
between 5 to 15 mg stephania extract, in another embodiment, about 5 mg stephania extract, in another embodiment, about 10 mg stephania extract, in another embodiment, about 15 mg stephania extract, in another embodiment, the stephania extract is selected from Stephania Tatrandra, Stephania Cepharantha Hayata, Stephania erecta Craib, Stephania rotunda Lour, Stephania rotunda Lour, Stephania sinica Diels and Stephania suberosa L. L. Forman; each represents a separate embodiment of this invention;
between 20-30 mg of β-sitosterole, in another embodiment, about 20 mg β-sitosterole, in another embodiment about 25 mg β-sitosterole, in another embodiment, about 30 mg β-sitosterole;
between 20-40 mg tocopherol (vitamin E), in another embodiment, about 20 mg tocopherol (vitamin E), in another embodiment, about 25 mg tocopherol (vitamin E), in another embodiment, about 30 mg tocopherol (vitamin E), in another embodiment, about 35 mg tocopherol (vitamin E), in another embodiment, about 40 mg tocopherol (vitamin E);
between 20-40 mg isoflavonoid, in another embodiment, about 20 mg isoflavonoid, in another embodiment, about 25 mg isoflavonoid, in another embodiment, about 30 mg isoflavonoid, in another embodiment, about 35 mg isoflavonoid, in another embodiment, about 40 mg isoflavonoid;
between 200-400 mg taurine, in another embodiment, about 200 mg taurine, in another embodiment, about 250 mg taurine, in another embodiment, about 300 mg taurine, in another embodiment, about 290 mg, in another embodiment, about 350 mg taurine, in another embodiment, about 400 mg taurine;
between 20-40 mg pyridoxine, in another embodiment, about 20 mg pyridoxine, in another embodiment, about 25 mg pyridoxine, in another embodiment, about 30 mg pyridoxine, in another embodiment, about 35 mg pyridoxine, in another embodiment, about 40 mg pyridoxine;
between 15-35 mg bambusa, in another embodiment, about 15 mg bambusa, in another embodiment, about 20 mg bambusa, in another embodiment, about 25 mg bambusa, in another embodiment, about 30 mg bambusa, in another embodiment, about 35 mg bambusa;
between 15-35 mg zinc picolinate, in another embodiment, about 15 mg zinc picolinate, in another embodiment, about 20 mg zinc picolinate, in another embodiment, about 25 mg zinc picolinate, in another embodiment, about 22 mg zinc picolinate, in another embodiment, about 30 mg zinc picolinate, in another embodiment, about 35 mg zinc picolinate;
between 10-30 mg cysteine, in another embodiment, about 10 mg cysteine, in another embodiment, about 15 mg cysteine,in another embodiment, about 20 mg cysteine, in another embodiment, about 25 mg cysteine, in another embodiment, about 30 mg cysteine;
between 10-30 mg thiamine chloride (vitamin B1), in another embodiment, about 10 mg thiamine chloride (vitamin B1), in another embodiment, about 15 mg thiamine chloride (vitamin B1), in another embodiment, about 20 mg thiamine chloride (vitamin B1), in another embodiment, about 25 mg thiamine chloride (vitamin B1); in another embodiment, about 30 mg thiamine chloride (vitamin B1);
between 10-30 mg pantothenic acid (vitamin B5), in another embodiment, about 10 mg pantothenic acid (vitamin B5), in another embodiment, about 15 mg pantothenic acid (vitamin B5),in another embodiment, about 20 mg pantothenic acid (vitamin B5), in another embodiment, about 25 mg pantothenic acid (vitamin B5), in another embodiment, about 30 mg pantothenic acid (vitamin B5),
between 10-30 mg riboflavin (vitamin B2), in another embodiment, about 10 mg riboflavin (vitamin B2), in another embodiment, about 15 mg riboflavin (vitamin B2), in another embodiment, about 20 mg riboflavin (vitamin B2), in another embodiment, about 25 mg riboflavin (vitamin B2), in another embodiment, about 30 mg riboflavin (vitamin B2);
between 1-5 mg copper, in another embodiment, about 1 mg copper, in another embodiment, about 1.5 mg copper, in another embodiment, about 2 mg copper, in another embodiment, about 2.5 mg copper, in another embodiment, about 3 mg copper, in another embodiment, about 3.5 mg copper, in another embodiment, about 4 mg copper, in another embodiment, about 4.5 mg copper, in another embodiment, about 5 mg copper;
between 0.1-1 mg cobalamin (vitamin B12), in another embodiment, about 0.1 mg cobalamin (vitamin B12), in another embodiment, about 0.2 mg cobalamin (vitamin B12), in another embodiment, about 0.3 mg cobalamin (vitamin B12), in another embodiment, about 0.4 mg cobalamin (vitamin B12), in another embodiment, about 0.5 mg cobalamin (vitamin B12), in another embodiment, about 0.6 mg cobalamin (vitamin B12), in another embodiment, about 0.7 mg cobalamin (vitamin B12), in another embodiment, about 0.8 mg cobalamin (vitamin B12), in another embodiment, about 1 mg cobalamin (vitamin B12);
between 0.1-1 mg biotine (vitamin H), in another embodiment, about 0.1 mg biotine (vitamin H), in another embodiment, about 0.2 mg biotine (vitamin H), in another embodiment, about 0.3 mg biotine (vitamin H), in another embodiment, about 0.4 mg biotine (vitamin H), in another embodiment, about 0.5 mg biotine (vitamin H), in another embodiment, about 0.6 mg biotine (vitamin H), in another embodiment, about 0.7 mg biotine (vitamin H), in another embodiment, about 0.8 mg biotine (vitamin H), in another embodiment, about 0.9 mg biotine (vitamin H), in another embodiment, about 1 mg biotine (vitamin H);
between 0.1-1 mg selenium, in another embodiment, about 0.1 mg selenium, in another embodiment, about 0.2 mg selenium, in another embodiment, about 0.3 mg selenium, in another embodiment, about 0.4 mg selenium, in another embodiment, about 0.5 mg selenium, in another embodiment, about 0.6 mg selenium, in another embodiment, about 0.7 mg selenium, in another embodiment, about 0.8 mg selenium, in another embodiment, about 0.9 mg selenium, in another embodiment, about 1 mg selenium;

In another embodiment, the nutrition supplement composition for female comprises:
10 mg stephania, wherein said stephania is selected from Stephania Tetrandra, Stephania Cepharantha Hayata, Stephania erecta Craib, Stephania rotunda Lour, Stephania rotunda Lour, Stephania sinica Diels or Stephania suberosa L. L. Forman; each represents a separate embodiment;
30 mg serenoa repens;
25 mg β-sitosterole
30 mg tocopherol (vitamin E);
290 mg taurine;
30 mg pyridoxine;
30 mg bambusa;
22 mg zinc picolinate;
20 mg cysteine;
20 mg thiamine (vitamin B1)
20 mg pantothenic acid (vitamin B5);
15 mg riboflavin (vitamin B2);
1.5 mg copper;
0.3 mg cobalamin (vitamin B12);
0.3 mg biotine (vitamin H);
0.1 mg selenium; and
30 mg isoflavonoid.

In one embodiment, the composition of this invention is administered with additional active ingredients. Non limiting examples of the additional active ingredients include antibacterial agent, antibiotic agent, antifungal agent, agent used for treating a dermatologic condition and any combination thereof.

In one embodiment the nutrition supplement composition of this invention further includes adjuvants, carriers, excipients, or stabilizers, and can be in solid or liquid form such as, tablets, capsules, pellets, powders, solutions, suspensions, or emulsions. Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

In one embodiment, the composition of this invention is administered on a daily base, or every other day. In another embodiment, the composition is a capsule, tablet or administered topically. In another embodiment, the composition is a cream or a gel. In one embodiment, the composition of this invention is administered for between 1 capsule/day to 5 capsules/day. In another embodiment, the composition of this invention is administered for 1 capsule/day. In another embodiment, the composition of this invention is administered for 2 capsule/day In another embodiment, the composition of this invention is administered for 3 capsule/day In another embodiment, the composition of this invention is administered for 4 capsule/day In another embodiment, the composition of this invention is administered for 5 capsule/day.

In one embodiment, the composition of this invention is used by men and women, aged above 18 yrs.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In another embodiment, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

In one embodiment, the nutrition supplement of this invention is orally administered, for example, with an inert diluent, or with an assimilable edible carrier, or they can be enclosed in hard or soft shell capsules, or they can be compressed into tablets, or they can be incorporated directly with the food of the diet. In another embodiment, the composition of this invention is a capsule.

Exemplary modes of administration include, without limitation, administering the compositions orally, topically, transdermally. In another embodiment, the composition of this invention is administered topically.

### Methods of Use

In one embodiment, this invention provides a method of treating, preventing, inhibiting or any combination thereof of hair loss for male and female, comprising administering a nutrition supplement composition of this invention. In another embodiment, this invention provides a method of treating, preventing, inhibiting or any combination thereof of hair loss for male, comprising administering a nutrition supplement composition of this invention.

In another embodiment, this invention provides a method of treating, preventing, inhibiting or any combination thereof of hair loss for female, comprising administering a nutrition supplement composition of this invention. In another embodiment, the nutrition supplement composition comprises serenoa repens, stephania, amino acid, vitamins and minerals. In another embodiment, the nutrition supplement composition comprises serenoa repens, cepharanthine, amino acid, vitamins and minerals. In another embodiment, the nutrition supplement composition comprises serenoa repens, amino acid, vitamins and minerals. In another embodiment, the nutrition supplement composition comprises serenoa repens, stephania extract, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and iron fumarate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and genisteine. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and ferrous bisglycinate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and isoflavonoid. In another embodiment, said stephania extract is selected from extracts of Stephania Tatrandra, Stephania Cepharantha Hayata, Stephania erecta Craib, Stephania rotunda Lour, Stephania rotunda Lour, Stephania sinica Diels and Stephania suberosa L. L. Forman. In another embodiment, the stephania extract is a Stephania Tetranda extract.

In one embodiment, this invention provides a method of treating alopecia, androgenic alopecia, alopecia areata, alopecia secondary to chemotherapy, alopecia secondary to radiation therapy, alopecia induced by scarring or alopecia induced by stress, comprising administering a nutrition supplement composition of this invention. In another embodiment, the nutrition supplement composition comprises serenoa repens, stephania, amino acid, vitamins and minerals. In another embodiment, the nutrition supplement composition comprises serenoa repens, cepharanthine, amino acid, vitamins and minerals. In another embodiment, the nutrition supplement composition comprises serenoa repens, amino acid, vitamins and minerals. In another embodiment, the nutrition supplement composition comprises serenoa repens, stephania extract, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and iron fumarate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and genisteine. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and ferrous bisglycinate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and isoflavonoid. In another embodiment, said stephania extract is selected from extracts of Stephania Tatrandra, Stephania Cepharantha Hayata, Stephania erecta Craib, Stephania rotunda Lour, Stephania rotunda Lour, Stephania sinica Diels and Stephania suberosa L. L. Forman. In another embodiment, the stephania extract is a Stephania Tetranda extract.

In one embodiment, this invention provides a method of hair growth stimulation in male and female, comprising administering a nutrition supplement composition of this invention. In one embodiment, this invention provides a method of hair growth stimulation in male, comprising administering a nutrition supplement composition of this invention. In one embodiment, this invention provides a method of hair growth stimulation in female, comprising administering a nutrition supplement composition of this invention. In another embodiment, the nutrition supplement composition comprises serenoa repens, stephania, amino acid, vitamins and minerals. In another embodiment, the nutrition supplement composition comprises serenoa repens, cepharanthine, amino acid, vitamins and minerals. In another embodiment, the nutrition supplement composition comprises serenoa repens, amino acid, vitamins and minerals. In another embodiment, the nutrition supplement composition comprises serenoa repens, stephania extract, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and iron fumarate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and genisteine. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and ferrous bisglycinate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and isoflavonoid. In another embodiment, said stephania extract is selected from extracts from Stephania Tatrandra, Stephania Cepharantha Hayata, Stephania erecta Craib, Stephania rotunda Lour, Stephania rotunda Lour, Stephania sinica Diels and Stephania suberosa L. L. Forman. In another embodiment, the stephania extract is a Stephania Tetranda extract.

In one embodiment, the composition of this invention improved the appearance of the hair, increased the amount of hair in the thinning area on the head. In another embodiment, the composition of this invention increased the growth of hair in the thinning area on the head.

In some embodiments, the composition of this invention is used for applications in or treating hair loss, alopecia, androgenic alopecia, alopecia areata, alopecia secondary to chemotherapy, alopecia secondary to radiation therapy, alopecia induced by scarring or alopecia induced by stress. In one embodiment, “hair loss”, or “alopecia”, refers to baldness as in the very common type of male-pattern baldness. Baldness typically begins with patch hair loss on the scalp and sometimes progresses to complete baldness and even loss of body hair. Hair loss affects both males and females. In another embodiment, the methods of this invention is used for increasing hair volume, and strengthening hair roots.

In one embodiment, this invention provides a method for replenishing the previously colored hair, comprising administering a nutrition supplement composition of this invention. In another embodiment, the nutrition supplement composition comprises serenoa repens, stephania, amino acid, vitamins and minerals. In another embodiment, the nutrition supplement composition comprises serenoa repens, cepharanthine, amino acid, vitamins and minerals. In another embodiment, the nutrition supplement composition comprises serenoa repens, amino acid, vitamins and minerals. In another embodiment, the nutrition supplement composition comprises serenoa repens, stephania extract, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and iron fumarate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and genisteine. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and ferrous bisglycinate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and isoflavonoid. In another embodiment, said stephania extract is selected from extracts of Stephania Tatrandra, Stephania Cepharantha Hayata, Stephania erecta Craib, Stephania rotunda Lour, Stephania rotunda Lour, Stephania sinica Diels and Stephania suberosa L. L. Forman. In another embodiment, the stephania extract is a Stephania Tetranda extract.

In another embodiment, the method of this invention turns gray hair to its previously colored hair in male and female. In another embodiment, the method of this invention turns gray hair to its previously colored hair in male. In another embodiment, the method of this invention turns gray hair to its previously colored hair in female.

In one embodiment, this invention provides a method of stimulation of healthy and strong nail growth, comprising administering a nutrition supplement composition of this invention. In another embodiment, the provided method stimulates a newer nail which replaces a previous nail. In another embodiment, the previous nail is fragile, fractured, friable, fungal infected or any combination thereof. In another embodiment, the cure rate of infected nails increases when an anti fungal powder was concomitantly applied with the compositions of this invention. As used herein, the term nail refers to both a toenail and an fingernail. In another embodiment, the nutrition supplement composition comprises serenoa repens, stephania, amino acid, vitamins and minerals. In another embodiment, the nutrition supplement composition comprises serenoa repens, cepharanthine, amino acid, vitamins and minerals. In another embodiment, the nutrition supplement composition comprises serenoa repens, amino acid, vitamins and minerals. In another embodiment, the nutrition supplement composition comprises serenoa repens, stephania extract, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and iron fumarate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and genisteine. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and ferrous bisglycinate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and isoflavonoid. In another embodiment, said stephania extract is selected from extracts from Stephania Tatrandra, Stephania Cepharantha Hayata, Stephania erecta Craib, Stephania rotunda Lour, Stephania rotunda Lour, Stephania sinica Diels and Stephania suberosa L. L. Forman. In another embodiment, the stephania extract is a Stephania Tetranda extract.

In one embodiment, this invention provides a method of stimulation of healthy and strong nail growth, comprising administering a nutrition supplement composition of this invention. In another embodiment, the provided method stimulates a newer nail which replaces a previous nail. In another embodiment, the previous nail is fragile, fractured, friable, fungal infected or any combination thereof. In another embodiment, the nutrition supplement composition comprises serenoa repens, stephania, amino acid, vitamins and minerals. In another embodiment, the nutrition supplement composition comprises serenoa repens, cepharanthine, amino acid, vitamins and minerals. In another embodiment, the nutrition supplement composition comprises serenoa repens, amino acid, vitamins and minerals. In another embodiment, the nutrition supplement composition comprises serenoa repens, stephania extract, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, cepharanthine, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and iron fumarate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and genisteine. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and ferrous bisglycinate. In another embodiment, the nutrition supplement composition comprises serenoa repens, stephania extract, β-sitosterole, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H), selenium and isoflavonoid. In another embodiment, said stephania extract is selected from extracts of Stephania Tatrandra, Stephania Cepharantha Hayata, Stephania erecta Craib, Stephania rotunda Lour, Stephania rotunda Lour, Stephania sinica Diels and Stephania suberosa L. L. Forman. In another embodiment, the stephania extract is a Stephania Tetranda extract.

In some embodiments, this invention provides a method of treating, preventing, inhibiting or any combination thereof of hair loss for male and female, a method of treating alopecia, androgenic alopecia, alopecia areata, alopecia secondary to chemotherapy, alopecia secondary to radiation therapy, alopecia induced by scarring or alopecia induced by stress, a method of hair growth stimulation in male and female, a method for replenishing the previously colored hair, a method of stimulation of healthy and strong nail growth or any combination thereof; comprising administering a nutrition supplement composition of this invention.

In one embodiment, the methods of this invention are useful in a subject, which is a human In another embodiment, the subject is a mammal In another embodiment the subject is an animal In one embodiment, the subject is male. In another embodiment, the subject is female.

In one embodiment, the methods of this invention make use of the nutrition supplement of this invention without having any side effects.

In one embodiment, the term “about”, refers to a deviance of between 0.0001-5% from the indicated number or range of numbers. In one embodiment, the term “about”, refers to a deviance of between 1-10% from the indicated number or range of numbers.

In one embodiment, the terms “a” or “an” as used herein, refer to at least one, or multiples of the indicated element, which may be present in any desired order of magnitude, to suit a particular application, as will be appreciated by the skilled artisan. In one embodiment, the term “an amino acid, a vitamin and a mineral refer to two or more kinds of amino acids, a vitamins and minerals.

The following examples are presented in order to more fully illustrate the preferred embodiments of the invention. They should in no way, however, be construed as limiting the broad scope of the invention.

### EXAMPLES

### Example 1

This example provides a capsule composition including dried herbs, amino acid, vitamins and minerals, comprising the following ingredients by weight:

### Formulation A (Male and Female):

| | % in Formula | Amount in |
|---|---|---|
| Component name | (w/w) | capsule (mg) |
| Serenoa repens | 12.18 | 30 |
| Cepharanthine | 0.81 | 2 |
| Tocopherol (vitamin E) | 12.18 | 30 |
| Taurine | 12.18 | 30 |
| Pyridoxine (Vitamin B6) | 12.18 | 30 |
| Bambusa | 10.15 | 25 |
| Zinc picolinate | 8.93 | 22 |
| Cysteine | 8.12 | 20 |
| (Vitamin B1) Thiamine | 8.12 | 20 |
| (Vitamin B5) Pantothenic | 8.12 | 20 |
| acid | | |
| Riboflavin (Vitamin B2) | 6.09 | 15 |
| Copper | 0.60 | 1.5 |
| (Vitamin B12) | 0.12 | 0.3 |
| Cobalamin | | |
| Biotine (Vitamin H) | 0.12 | 0.3 |
| Selenium | 0.04 | 0.1 |
| Total | 100.0 | 246.2 |

### Example 2

This example provides a capsule composition including dried herbs, amino acid, vitamins and minerals, comprising the following ingredients by weight:

### Formulation B: (Male and Female)

| | | Amount |
|---|---|---|
| | % in | in |
| | Formula | capsule |
| Component name | (w/w) | (mg) |
| Serenoa repens | 49 | 300 |
| Stephania | 16.3 | 100 |
| cepharantha hayata | | |
| Tocopherol (vitamin | 4.9 | 30 |
| E) | | |
| Taurine | 4.9 | 30 |
| Pyridoxine (Vitamin | 4.9 | 30 |
| B6) | | |
| Bambusa | 4.1 | 25 |
| Zinc picolinate | 3.6 | 22 |
| Cysteine | 3.3 | 20 |
| (Vitamin B1) | 3.3 | 20 |
| Thiamine | | |
| (Vitamin B5) | 3.3 | 20 |
| Pantothenic acid | | |
| Riboflavin (Vitamin | 2.44 | 15 |
| B2) | | |
| Copper | 0.24 | 1.5 |
| (Vitamin B12) | 0.05 | 0.3 |
| Cobalamin | | |
| Biotine (Vitamin H) | 0.05 | 0.3 |
| Selenium | 0.02 | 0.1 |
| Total | 100% | 614.2 |

### Example 3

This example provides a capsule composition for male treatment including dried herbs, amino acid, vitamins and minerals, comprising the following ingredients by weight:

### Formulation C: (Male):

| | | Amount |
|---|---|---|
| | % in | in |
| | Formula | capsule |
| Component name | (w/w) | (mg) |
| Serenoa repens | 5.14 | 30 |
| Stephania Tetrandra | 1.71 | 10 |
| β-sitosterole | 8.56 | 50 |
| Tocopherol (vitamin | 5.14 | 30 |
| E) | | |
| Taurine | 51.35 | 300 |
| Pyridoxine (Vitamin | 5.14 | 30 |
| B6) | | |
| Bambusa | 3.42 | 20 |
| Zinc picolinate | 3.77 | 22 |
| Cysteine | 3.42 | 20 |
| (Vitamin B1) | 3.42 | 20 |
| Thiamine | | |
| (Vitamin B5) | 3.42 | 20 |
| Pantothenic acid | | |
| Riboflavin (Vitamin | 2.57 | 15 |
| B2) | | |
| Copper | 0.26 | 1.5 |
| (Vitamin B12) | 0.05 | 0.3 |
| Cobalamin | | |
| Biotine (Vitamin H) | 0.05 | 0.3 |
| Selenium | 0.02 | 0.1 |
| Iron fumarate | 2.57 | 15 |
| Total | 100% | 584.2 |

### Example 4

This example provides a capsule composition for male treatment including dried herbs, amino acid, vitamins and minerals, comprising the following ingredients by weight:

### Formulation D: (Male):

| | | Amount |
|---|---|---|
| | % in | in |
| | Formula | capsule |
| Component name | (w/w) | (mg) |
| Serenoa repens | 5.10 | 30 |
| Stephania Tetrandra | 1.70 | 10 |
| β-sitosterole | 8.50 | 50 |
| Tocopherol (vitamin | 5.10 | 30 |
| E) | | |
| Taurine | 49.20 | 290 |
| Pyridoxine (Vitamin | 5.10 | 30 |
| B6) | | |
| Bambusa | 5.94 | 35 |
| Zinc picolinate | 3.71 | 22 |
| Cysteine | 3.40 | 20 |
| (Vitamin B1) | 3.40 | 20 |
| Thiamine | | |
| (Vitamin B5) | 3.40 | 20 |
| Pantothenic acid | | |
| Riboflavin (Vitamin | 2.54 | 15 |
| B2) | | |
| Copper | 0.25 | 1.5 |
| (Vitamin B12) | 0.05 | 0.3 |
| Cobalamin | | |
| Biotine (Vitamin H) | 0.05 | 0.3 |
| Selenium | 0.02 | 0.1 |
| Ferrous bisglycinate | 2.54 | 15 |
| Total | 100% | 589.2 |

### Example 5

This example provides a capsule composition for female treatment including dried herbs, amino acid, vitamins and minerals, comprising the following ingredients by weight:

### Formulation E: (Female):

| | | Amount |
|---|---|---|
| | % in | in |
| | Formula | capsule |
| Component name | (w/w) | (mg) |
| Serenoa repens | 5.22 | 30 |
| StephaniaTetrandra | 1.74 | 10 |
| β-sitosterole | 4.34 | 25 |
| Tocopherol (vitamin | 5.22 | 30 |
| E) | | |
| Taurine | 52.25 | 300 |
| Pyridoxine (Vitamin | 5.14 | 30 |
| B6) | | |
| Bambusa | 3.48 | 20 |
| Zinc picolinate | 3.83 | 22 |
| Cysteine | 3.48 | 20 |
| (Vitamin B1) | 3.48 | 20 |
| Thiamine | | |
| (Vitamin B5) | 3.48 | 20 |
| Pantothenic acid | | |
| Riboflavin (Vitamin | 2.61 | 15 |
| B2) | | |
| Copper | 0.26 | 1.5 |
| (Vitamin B12) | 0.05 | 0.3 |
| Cobalamin | | |
| Biotine (Vitamin H) | 0.05 | 0.3 |
| Selenium | 0.02 | 0.1 |
| Genisteine | 5.22 | 30 |
| Total | 100% | 574.2 |

### Example 6

This example provides a capsule composition for female treatment including dried herbs, amino acid, vitamins and minerals, comprising the following ingredients by weight:

### Formulation F: (Female):

| | | Amount |
|---|---|---|
| | % in | in |
| | Formula | capsule |
| Component name | (w/w) | (mg) |
| Serenoa repens | 5.22 | 30 |
| Stephania Tetrandra | 1.72 | 10 |
| β-sitosterole | 4.35 | 25 |
| Tocopherol (vitamin | 5.22 | 30 |
| E) | | |
| Taurine | 50.50 | 290 |
| Pyridoxine (Vitamin | 5.22 | 30 |
| B6) | | |
| Bambusa | 5.22 | 30 |
| Zinc picolinate | 3.80 | 22 |
| Cysteine | 3.5 | 70 |
| (Vitamin B1) | 3.5 | 20 |
| Thiamine | | |
| (Vitamin B5) | 3.5 | 20 |
| Pantothenic acid | | |
| Riboflavin (Vitamin | 2.61 | 15 |
| B2) | | |
| Copper | 0.30 | 1.5 |
| (Vitamin B12) | 0.05 | 0.3 |
| Cobalamin | | |
| Biotine (Vitamin H) | 0.05 | 0.3 |
| Selenium | 0.02 | 0.1 |
| Isoflavonoid | 5.22 | 30 |
| Total | 100% | 574.2 |

### Example 5

### Formulation B for Treating Androgenic Alopecia in Men

A study design was performed including double blinded, randomized, placebo controlled clinical trial for six months. The study population included 33 healthy male subjects at the age of 18-40 with mild to moderate androgenic alopecia. Randomized study was at 2:1 ratio (23 active and 10 placebo), wherein only 29 where analyzed (3 lost follow up, 1 protocol deviation). Each subject received 90 pills for 6 months.

Demographics: The average age was 30.7±5.1; average weight: 79.8±13.4; diet: 31 of 33 omnivorous, 2 pescetarian; smoking: 25 never smoked; 8 previously/currently smoking; use of medication: 5 were using medications for stress/anxiety.

Hair characteristics: Hemilton-Norwood classification: 2-5; age hair loss first seen: 22.8±4.2; white hair: none=21, few=11, many=1; hair loss treatment: Finasteride=4; Minoxydil =5; other=4; none=20.

Dandruff: none.

### Methods

The results were determined using global photographic evaluation, hair count, subjective evaluation, safety and personal experience.

### Photographic Method

Photographs taken by the OmniaSystem (Canfield), specialized for esthetic and dermatological indications.

Global photographs of midline and vertex of baseline vs 6 months were used for this evaluation. The regions assessed were the vertex and superio-frontal regions, as well as overall.

The validated improvement scale is used in all clinical hair studies and spans from −3=greatly decreased to +3 greatly increased.

Accountability: 2 subjects had drastic hair cuts and were therefore not included in this analysis.

### Hair Count Method

The target area of hair count (TAHC) was selected from the anterior edge of the balding area, hairs were clipped, and the area was marked with a small medical tattoo.

A macro-photograph of the TAHC was taken. The photograph included a reference scale.

Hairs were manually counted in a 1 cm² circular area of clipped hair at baseline, 3 months, and 6 months.

Hair counters (n=2) were blinded to group allocation.

White/blond hairs were not counted.

Hair counts included terminal hairs. In several cases it was possible to differentiate between terminal and vellus hairs.

### Results

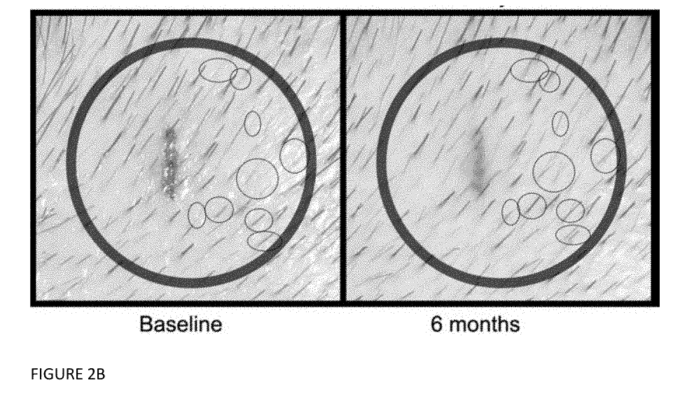

It was found that the vellus hair counts are decreased, while terminal hairs counts are increased (FIG. 2A). In the macro-photograph it can be seen that the terminal hairs replace vellus hairs after 6 months of treatment (FIG. 2B).

Subjects of the active group felt significantly more satisfied with all aspects of hair appearance, whereas the placebo group did not.

This included:

Overall appearance (p*=0.002 vs 0.14, active vs placebo by Wilcoxon sign ranks test)

Appearance of “Thinning Areas” (TAs) (p=0.006 vs 0.22);

The amount of scalp in the TAs (p=0.025 vs p=1);

The amount of hair in the TAs (p=0.028 vs p=1);

The growth of hair in the TAs (p=0.002 vs p=0.66);

The result is considered significant when the p value <0.05.

No adverse events were reported in the active group.

### Example 6

### Formulation C and D for Treating Androgenic Alopecia in Men and Women

The study population included 105 healthy male subjects at the age of 23-67 with mild to moderate androgenic alopecia and 96 healthy female subjects at the age of 22-78. Of the total 201 men and women group, 54% were advised to stop formulation B and continue with the relevant gender-specific formula (C-men, D-women) whereas 46% have begun with the relevant gender-specific formula (C-men, D-women) without using formula B prior to that. Each subject that used formula C or D received one pill for a day.

### Results

It was found that in the group that used initially formula B and then continued with formula C or D, no setbacks of any kind were reported. In the other group that begun with formula C or D, hair lost stopped within 3-5 weeks of usage and the hair regrowth started within the same period of time. In 22% of the patients they reported a significant improvement. All patients reported an increase in general wellbeing.

### Example 7

### Nail Growth Using the Composition of this Invention

One of the formulation described in he above examples (formulations A-F) were taken by men or women respectively, once a day for two weeks. A fast growth of new, healthy and strong nails that replaced previous fragile, fractured and friable nails was reported following these two weeks of administration. It was also observed that the new growing nails succeeded in replacing fungal infected toes nails.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1-38. (canceled)

39. A nutrition supplement composition comprising serenoa repens, stephania tetrandra, at least one amino acid, at least one vitamin and at least one mineral.

40. The composition of claim 39, further comprising phytosterol.

41. The composition of claim 40, further comprising isoflavonoid.

42. The composition of claim 40, further comprising taurine and bambusa.

43. The composition of claim 41, further comprising taurine and bambusa.

44. The composition of claim 39, wherein said composition comprises serenoa repens, stephania tetrandra, tocopherol (vitamin E), taurine, pyridoxine (vitamin B6), bambusa, zinc picolinate, cysteine, Thiamine (vitamin B1), pantothenic acid (vitamin B5), riboflavin (vitamin B2), copper, cobalamin (vitamin B12), biotine (Vitamine H) and selenium.

45. The composition of claim 44, further comprising β-sitosterole and iron fumarate.

46. The composition of claim 44, further comprising β-sitosterole and Genistin.

47. The composition of claim 44, further comprising β-sitosterole and ferrous bisglycinate.

48. A method of treating, preventing, inhibiting, or any combination thereof hair loss in male and female comprising administering a composition according to claim 39.

49. A method of stimulating hair growth in male and female comprising administering a composition according to claim 39.

50. A method of replenishing the previously colored hair in male and female comprising administering a composition according to claim 39.

51. A method of stimulating healthy and strong finger nail growth in male and female comprising administering a composition according to claim 39.

52. The method of claim 51, wherein said nail growth results in a new grown nail; said new grown nail replaces a previous nail; and said previous nail is fragile, fractured, friable, fungal infected or any combination thereof.

53. A method of treating, preventing, inhibiting or any combination thereof hair loss, stimulating hair growth, replenishing the previously colored hair, stimulating healthy and strong finger nail growth or any combination thereof in a subject, comprising administering a composition according to claim 39.

54. The method of claim 53, wherein the subject is a male or a female.
